(19) United States
(12) Patent Application Publication
(10) Pub. No.: US 2023/0372437 A1
(43) Pub. Date: Nov. 23, 2023

(54) COMPOSITIONS AND METHODS FOR THE TREATMENT OF HAIR LOSS AND OTHER CONDITIONS

(71) Applicant: ProTransit Nanotherapy LLC, Omaha, NE (US)

(72) Inventors: Gary L. Madsen, Omaha, NE (US); Philippe Burnham, Simi Valley, CA (US); Christopher L. Porter, Omaha, NE (US)

(21) Appl. No.: 18/246,572

(22) PCT Filed: Sep. 24, 2021
(86) PCT No.: PCT/US21/51910

§ 371 Date: Mar. 24, 2023

Related U.S. Application Data
(60) Provisional application No. 63/082,613, filed on Sep. 24, 2020.

(51) Int. Cl. A61K 38/13; A61K 31/473; A61P 17/14; A61K 8/64; A61K 8/49; A61Q 5/06

(52) U.S. Cl. CPC A61K 38/13; A61K 31/473; A61P 17/14; A61K 8/64; A61K 8/4926; A61Q 5/06

(57) ABSTRACT

Compositions and methods for the treatment of hair loss and/or gray hair are provided.

This application is claims priority under 35 U.S.C. § 119(e) to U.S. Provisional Patent Application No. 63/082,613, filed on Sep. 24, 2020. The foregoing application is incorporated by reference herein.

### FIELD OF THE INVENTION

The present invention relates to the treatment, inhibition, and/or prevention of hair loss and other conditions including gray hair. Compositions and methods are provided for promoting hair regrowth and/or thickening.

### BACKGROUND OF THE INVENTION

The hair regrowth marketplace is large and growing with 35 million men and 30 million women in the U.S. experiencing visible hair loss in 2020. Furthermore, the marketplace is also plagued with off-label compounding and the marketing of single and multiple ingredient formulations with unproven safety and efficacy, as well as many compositions making unsubstantiated claims for hair regrowth.

Current non-surgical approaches are sub-optimal due to their lack of being able to significantly penetrate the protective stratum corneum, effectively influence the dermal stems cells, and restore senescent hair follicles. Indeed, minoxidil is well known to be poorly absorbed and orally administered finasteride is well known to causes systemic side effects and be unsafe to male fetuses in premenopausal women. While surgical approaches have improved, they are prohibitively expensive for a large portion of the market (typically greater than $10,000 for a hair transplant). In view of the foregoing, it is clear that improved methods for restoring hair regrowth are needed.

### SUMMARY OF THE INVENTION

In accordance with the instant invention, methods of treating, inhibiting, and/or preventing hair loss and/or related disorders are provided. In a particular embodiment, the method comprises topically administering at least one nanoparticle (e.g., to the skin (e.g., scalp)) of the subject, wherein the nanoparticle comprises at least one biodegradable polymer and at least one hair regrowth agent. The nanoparticles may be administered before and/or after hair loss (e.g., the nanoparticles can be administered as a preventive measure and/or administered to promote hair regrowth and/or to maintain hair count). The hair regrowth agent may be, without limitation, minoxidil, finasteride, cyclosporine-A, and/or bimatoprost. The biodegradable polymer may be, for example, poly (lactide-co-glycolide) (e.g., poly(DL-lactide-co-glycolide)), polylactide, or derivatives thereof. The nanoparticle may further comprise at least one plasticizer (e.g., dimethyl tartrate). The methods of the instant invention may also comprise the administration of at least one other agent (e.g., a therapeutic agent, a repigmentation agent (e.g., an antioxidant (e.g., a nanoparticle comprising an antioxidant (e.g., antioxidant enzyme such as catalase))), or additional hair regrowth agent). The nanoparticles may be administered using a suitable carrier for topical application (e.g., lotion, cream, haircare product, etc.). The carrier may be water-in-oil (w/o), oil-in-water (o/w), water-in-oil-in-water (w/o/w), oil-in-water-in-oil (o/w/o), oil-in-oil (o/o), anhydrous, or a combination of different bases. The carrier may be polymeric or natural hydrogel or films, which may be prepared before application or form in situ following application. The carrier may contain a skin permeation enhancer (e.g., surfactants (e.g., polysorbates, CTAB, DMAB), solvents (e.g., benzyl alcohol, isopropyl alcohol)), moisturizer, lubricant, color, dye, fragrance, etc.

In accordance with another aspect of the instant invention, topical compositions are provided which are well-suited for the delivery of compounds to the skin (e.g., scalp), particularly beneath the stratum corneum and in hair follicles. In a particular embodiment, the topical composition comprises at least one carrier (e.g., a carrier acceptable for topical delivery (e.g., a pharmaceutically and/or cosmetically acceptable carrier)) and nanoparticles comprising at least one hair regrowth agent.

### BRIEF DESCRIPTIONS OF THE DRAWING

FIG. 1 provides a graph of the in vitro release of finasteride from nanoparticles. Dashed line indicates immediate release. Particles show measurable release of finasteride through at least day 4.

FIG. 2 provides a graph of the in vitro release of cyclosporine-A from nanoparticles. Particles show measurable release of drug through at least day 17.

FIG. 3 provides images of hair regrowth after one month of finasteride loaded nanoparticles to human scalp suffering from alopecia. Photo on the left shows the baseline amount of hair, with circled numbers indicating individual hairs that were no longer present at month 1. Photo on the right shows scalp at 1 month with circled numbers indicating hairs that appeared after start of application.

### DETAILED DESCRIPTION OF THE INVENTION

Topical minoxidil has been the mainstay treatment for androgenetic alopecia for over 30 years and is used off-label for other hair loss conditions (e.g., alopecia areata and scarring alopecia) and to improve body hair regrowth including the eyebrows and beard. Minoxidil is a potent vasodilator and when used twice daily (e.g., as a 2% or 5% solution) for at least 3-4 months, has been shown to restore some hair regrowth in a large percentage of users over a 9 to 12-month time frame. Only about 10% of users experience dense hair regrowth and most only see a slowing down of hair loss or a modest increase of 6-9 hairs/cm² of scalp). Results are quite variable from patient to patient in terms of time to regrow hair and overall improvement of hair regrowth. This wide range of outcomes is likely due to the inherent poor absorption of minoxidil. Indeed, only about 1.7% of the topically applied minoxidil is absorbed through a normal scalp. Moreover, in addition to the poor absorption through the skin, transfollicular absorption of minoxidil is hampered by the frequent presence of a sebum plug. Increased absorption is associated with drug concentration, frequency of drug application, and damage to the barrier function of the stratum corneum. Systemic doses in the range of 2.4 to 5.4 mg/day can be achieved if application is made to the entire scalp.

Currently, liquid topical minoxidil formulations contain propylene glycol to enhance minoxidil penetration. However, propylene glycol is commonly associated with skin irritation. Despite the inclusion of propylene glycol, minoxidil formulations still have poor skin and transfollicular penetration, thereby leading to their low efficacy rates.

Improved topical drug delivery may be achieved by using nanoparticles. The small size and increased surface area of nanoparticles enables a close and extended contact with the stratum corneum. Moreover, nanoparticles allow for controlled drug release which will lead to deeper penetration of the drug into skin strata while minimizing both the required drug dosage and drug losses. The use of nanoparticles will reduce topical adverse effects while increasing therapeutic efficacy. The drug-loaded nanoparticles can also penetrate the sebum plug more effectively than aqueous formulation. As such, this will increase drug penetration and deposition into deeper skin layers/strata and increase accumulation in hair follicles which can function as drug reservoirs for further drug delivery.

Poly(DL-lactide-co-glycolide) (PLGA) nanoparticles and poly(L-lactide-co-glycolide) (PLLGA) nanoparticles encapsulating minoxidil have been synthesized (Takeuchi et al., Bio-Medical Materials and Engineering (2018) 29:217-228). The PLLGA nanoparticles had a more sustained release than PLGA nanoparticles. PLLGA nanoparticles were determined to deliver 3.1 times more minoxidil in the stratum corneum and 2.5 times more minoxidil into the follicle bulb compared to a minoxidil aqueous solution. However, despite the more sustained release compared to PLGA nanoparticles, PLLGA nanoparticles still released 68% of the encapsulated minoxidil in only 8 hours.

Additionally, PLGA microspheres encapsulating finasteride have been synthesized (Kim, et al., Int. J. Mol. Med. (2019) 43(6):2409-2419). Indeed, these microspheres, when applied via subcutaneous injection, were able to improve hair regrowth in a testosterone induced androgenic alopecia mouse model. However, topical administration of these microspheres was not reported.

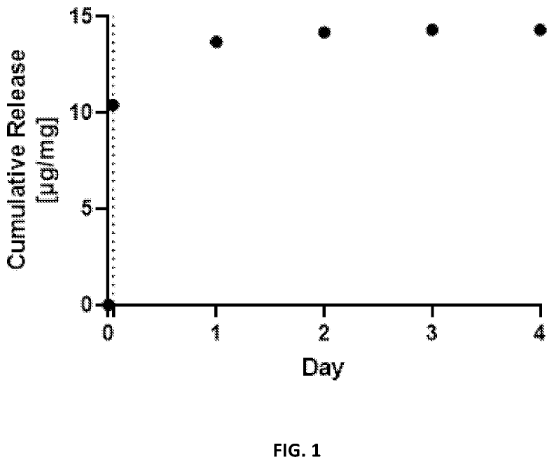

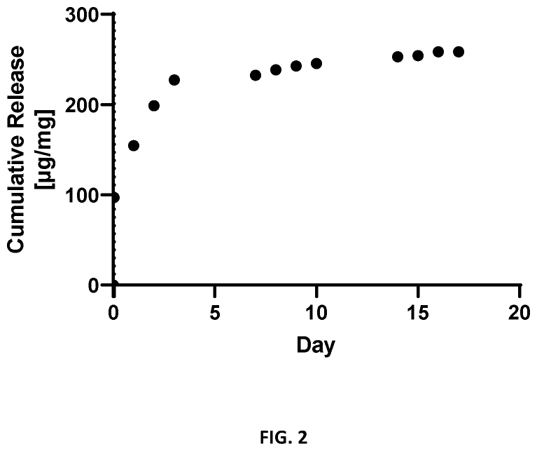

The nanoparticles of the instant invention allow for the deep penetration of compounds within the skin, such as the epidermis. After administration, the nanoparticles of the instant invention allow for the gradual release of the active ingredient to safely and predictably optimize delivery of a targeted drug throughout the skin (see, e.g., FIG. 1 and FIG. 2). In certain embodiments, the nanoparticles of the instant invention penetrate into the hair follicle bulb, which can serve as a secondary delivery system or “drug depot.” The release of the encapsulated active ingredients from the hair follicle bulb can further promote the gradual release of the active ingredients throughout the skin over time.

In accordance with one aspect of the instant invention, nanoparticles (sometimes referred to herein as Pro-NP™) which encapsulate at least one hair regrowth agent and/or antioxidant are provided. The nanoparticles of the instant invention stabilize the encapsulated compound, allow the penetration of the agent through the skin layers and into hair follicles (e.g., past the sebum plug), and deliver hair regrowth agent over a sustained period of time. The nanoparticles of the instant invention can also be completely metabolized by the body in a non-toxic manner. In a particular embodiment, the nanoparticles of the instant invention congregate/concentrate in the base/bulb of the hair follicle and release the encapsulated hair regrowth agent over a sustained period of time (e.g., up to 95 hours or more).

The nanoparticles of the instant invention provide a superior drug delivery system that is able to: (1) increase the hair regrowth agent's stability, (2) improve the skin pharmacokinetic and/or pharmacodynamic profiles of the hair regrowth agent, (3) increase the permeation and formation of skin depots in the stratum corneum, epidermis, and/or follicular bulb, (4) promote therapeutic adherence, and/or (5) decrease toxicity and/or treatment resistance to the hair regrowth agent.

In accordance with the instant invention, methods of delivering a compound to the hair follicle (e.g., transfollicular) are provided. In a particular embodiment, the method results in the concentration of the nanoparticles within the hair follicle (e.g., a greater number of nanoparticles within the hair follicle than predicted by an even distribution across the skin). The methods of the instant invention comprise administering (particularly topically) at least one nanoparticle of the instant invention (or a composition comprising at least one nanoparticle) comprising or encapsulating the compound to a subject. In a particular embodiment, the subject is a male. In a particular embodiment, the subject is female (e.g., a post-menopausal woman). In a particular embodiment, the methods deliver the compound across the sebum plug. In a particular embodiment, the compound is a hair regrowth agent. In a particular embodiment, the compound is repigmentation agent or an antioxidant. In certain embodiments, the compound is a small molecule. In certain embodiments, the compound is an enzyme.

In accordance with another aspect of the instant invention, methods of treating, inhibiting, and/or preventing hair loss and/or related disorders are provided. Examples of hair loss disorders include, without limitation: alopecia, alopecia areata, androgenetic alopecia (alopecia androgenetica), hypotrichosis (e.g., of the eyelash or eyebrow) and hair miniaturization. The methods of the instant invention comprise administering (particularly topically) at least one nanoparticle of the instant invention (or a composition comprising at least one nanoparticle) comprising or encapsulating a hair regrowth agent to a subject. The method may comprise topically applying the nanoparticle and/or composition of the instant invention to the area of skin (e.g., scalp) to be treated (e.g., in need of hair regrowth and/or maintenance). In a particular embodiment, the subject is a male. In a particular embodiment, the subject is female (e.g., a post-menopausal woman). In a particular embodiment, the hair regrowth agent is a small molecule. The methods may further comprise the administration of at least one other therapeutic agent for the treatment, inhibition, or prevention of hair loss and/or related disorders (e.g., graying). For example, the method may further comprise the administration of a repigmentation agent (e.g., an antioxidant (e.g., antioxidant enzyme such as catalase)). In certain embodiments, the additional therapeutic agent is contained within the same nanoparticle as the hair regrowth agent and/or in separate nanoparticles from the hair regrowth agent nanoparticles. The additional therapeutic agent (encapsulated in a nanoparticle or not) may be administered in the same composition as the hair regrowth agent nanoparticles. The additional therapeutic agent (encapsulated in a nanoparticle or not) may be administered in a separate composition from the hair growth agent nanoparticles of the instant invention. The compositions may be administered at the same time or at different times (e.g., sequentially).

In accordance with another aspect of the instant invention, methods of reducing and/or preventing gray hair are provided. The methods of the instant invention comprise administering (particularly topically) at least one nanoparticle of the instant invention (or a composition comprising at least one nanoparticle) comprising or encapsulating a repigmentation agent to a subject. The method may comprise topically applying the nanoparticle and/or composition of the instant invention to the area of skin (e.g., scalp) to be treated (e.g., in need of hair repigmentation). The subject may be male or female. In a particular embodiment, the repigmentation agent is an antioxidant (e.g., as described herein). In a particular embodiment, the antioxidant is an antioxidant enzyme, particularly catalase and/or methionine sulfoxide reductase (e.g., of mammalian, particularly human, origin). In a particular embodiment, the antioxidant is L-methionine. The presence of gray hair is usually due to an accumulation of H₂O₂ in the skin or scalp, thereby effectively “bleaching” hair (Wood et al. (2009) FASEB J., 23:2065-2075). Elimination and/or reduction of the H₂O₂ will reduce the amount of gray hair. Examples of repigmentation agents are provided in Yale et al. (Skin Appendage Disorder. (2020) 6:1-10), incorporated by reference herein—particularly those agents provided in Table 1. The methods may further comprise the administration of at least one other agent for the reduction and/or prevention of gray hair and/or at least one other therapeutic agent for the treatment, inhibition, or prevention of hair loss and/or related disorders. For example, the method may further comprise the administration of a hair regrowth agent. In certain embodiments, the additional therapeutic agent is contained within the same nanoparticle as the repigmentation agent and/or in separate nanoparticles from the repigmentation agent nanoparticles. The additional therapeutic agent (encapsulated in a nanoparticle or not) may be administered in the same composition as the repigmentation agent nanoparticles. The additional agent (encapsulated in a nanoparticle or not) may be administered in a separate composition from the repigmentation nanoparticles of the instant invention. The compositions may be administered at the same time or at different times (e.g., sequentially).

The nanoparticles of the instant invention comprise at least one polymer and at least one encapsulated compound. Generally, the nanoparticle ranges in size between 1 nm and 1000 nm. In certain embodiments, the nanoparticle ranges in size between 1 nm and about 600 nm, between 1 nm and about 500 nm, between 1 nm and about 350 nm, between 1 nm and about 250 nm, between about 100 nm and about 600 nm, between about 300 nm and about 600 nm, between about 350 nm and about 550 nm, between about 150 nm and about 350 nm, between about 400 and about 500 nm, or between about 200 nm and about 300 nm. While the instant invention generally describes the use of small molecules in the nanoparticles, it is also within the scope of the instant invention to use other therapeutic agents or compounds of interest in the nanoparticles. Such agents or compounds include, without limitation, polypeptides, proteins, peptides, glycoproteins, nucleic acids (DNA, RNA, oligonucleotides, plasmids, siRNA, etc.), synthetic and natural drugs, polysaccharides, lipids, and the like.

In a particular embodiment, the polymer of the nanoparticles is a biocompatible and biodegradable polymer. The polymer may be a homopolymer or a copolymer. In a particular embodiment, the polymer is a copolymer. The polymer may be hydrophobic, hydrophilic, or amphiphilic. If the polymer is a copolymer, it may be a random, diblock, triblock, or multiblock copolymer. In a particular embodiment, the segments of the block copolymer comprise about 10 to about 500 repeating units, about 20 to about 300 repeating units, about 20 to about 250 repeating units, about 20 to about 200 repeating units, or about 20 to about 100 repeating units. Suitable polymers include, without limitation: poly(lactide-co-glycolides) (e.g., PLGA, PLLGA, etc.), poly(lactic acid), poly(alkylene glycol), polybutylcyanoacrylate, poly(methylmethacrylate-co-methacrylic acid), poly-allylamine, polyanhydride, polyhydroxybutyric acid, polyorthoesters, and the like. In particular embodiments, a nanoparticle is composed of a copolymer comprising lactic acid and glycolic acid. In particular embodiments, a nanoparticle is composed of a copolymer (e.g., a block copolymer) comprising at least one poly(lactic acid) segment and at least one poly(glycolic acid) segment. In a particular embodiment, the polymer is a poly (lactide-co-glycolide), particularly poly (D,L-lactide-co-glycolide) (PLGA). Examples of biocompatible polymers include, without limitation: natural or synthetic polymers such as polystyrene, polylactic acid, polyketal, butadiene styrene, styreneacrylic-vinyl terpolymer, polymethylmethacrylate, polyethylmethacrylate, polyalkylcyanoacrylate, styrene-maleic anhydride copolymer, polyvinyl acetate, polyvinylpyridine, polydivinylbenzene, polybutyleneterephthalate, acrylonitrile, vinylchloride-acrylates, polycaprolactone, poly(alkyl cyanoacrylates), poly(lactic-co-glycolic acid), and the like. Examples of natural polymers include polypeptides including those modified non-peptide components, such as saccharide chains and lipids; nucleotides; sugar-based biopolymers such as polysaccharides; cellulose; carbohydrates and starches; dextrans; lignins; polyamino acids; adhesion proteins; lipids and phospholipids (e.g., phosphorylcholine).

The nanoparticles of the present invention can further contain a polymer that affects the charge or lipophilicity or hydrophilicity of the particle. Any biocompatible polymer can be used for this purpose, including but not limited to, poly(vinyl alcohol).

The nanoparticles of the present invention can further comprise a plasticizer. The plasticizer may facilitate sustained release of the encapsulated compound by maintaining the structure of the nanoparticle. A plasticizer may be added to the nanoparticles to maintain the glass transition temperature above 37° C. despite a decline in molecular weight of the polymer with time. Without being bound by theory, the addition of the plasticizer allows for pores in the nanoparticle to remain open and facilitate a continuous release of the encapsulated compound. Suitable plasticizers are generally inert, non-toxic, and biocompatible. Plasticizers include, without limitation, triethyl citrate (e.g., Citroflex®, Morflex Inc., Greensboro, N.C.), glyceryl triacetate (e.g., triacetin), L-tartaric acid dimethyl ester (dimethyl tartrate, DMT), benzoates (e.g. terephthalates such as dioctyl terephthalate/DEHT,1,2-cyclohexane dicarboxylic acid diisononyl ester (Hexamoll® DINCH®), epoxidized vegetable oils, alkyl sulphonic acid phenyl ester (ASE), sulfonamides (e.g. N-ethyl toluene sulfonamide (o/p ETSA), ortho and para isomers, N-(2-hydroxypropyl) benzene sulfonamide (HP BSA), N-(n-butyl) benzene sulfonamide (BBSA-NBBS)), organophosphates (e.g., tricresyl phosphate (TCP), tributyl phosphate (TBP)), glycols/polyethers, triethylene glycol (e.g., dihexanoate (3G6, 3GH), tetraethylene glycol diheptanoate (4G7)), polymeric plasticizer (e.g. polybutene), and bio-based plasticizers. Bio-based plasticizers may have better biodegradability and fewer biochemical effects and include, without limitation: acetylated monoglycerides, alkyl citrates, triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), andrimethyl citrate (TMC). In a particular embodiment, the nanoparticles comprise the plasticizer triethyl citrate. In a particular embodiment, the nanoparticles comprise the plasticizer dimethyl tartrate (DMT) or tartaric acid. The amount of plasticizer employed in a nanoparticle can range from about 5 to about 40 weight percent of the nanoparticle, particularly from about 10 to 20 weight percent of the nanoparticle (e.g., compared to the weight of polymer or the total nanoparticle). In particular embodiments, the plasticizer encompasses about 10 weight percent of the nanoparticle.

The nanoparticles of the instant invention may also comprise a surfactant or emulsifier (e.g., polyvinyl alcohol) to facilitate their dispersion and stability in the topical formulation. In certain embodiments, the surfactant or emulsifier coats the nanoparticle. These surface-associated surfactants/emulsifier can be anionic (e.g., sodium dodecyl sulfate, sodium dodecyl benzene sulfonate, sodium laureth sulfate, sodium lauroyl sarcosinate, sodium myreth sulfate, sodium pareth sulfate, sodium stearate, etc.), neutral (e.g., ethoxylated aliphatic alcohol, polyoxyethylene surfactants, carboxylic esters, polyethylene glycol esters, anhydrosorbitol ester and ethoxylated derivatives thereof, glycol esters of fatty acids, carboxylic amides, monoalkanolamine condensates, polyoxyethylene fatty acid amides), or cationic (e.g., quaternary ammonium salts, amines with amide linkages, polyoxyethylene alkyl and alicyclic amines, N,N,N′,N′ tetrakis substituted ethylenediamines, 2-alkyl 1-hydroxethyl 2-imidazolines); amphoteric type (e.g., amphoteric surfactants contains both an acidic and a basic hydrophilic moiety in their surface, N-coco 3-aminopropionic acid/sodium salt, N-tallow 3-iminodipropionate disodium salt, N-carboxymethyl N dimethyl N-9 octadecenyl ammonium hydroxide, N-cocoamidethyl-N-hydroxyethylglycine sodium salt. In a particular embodiment, the surfactant or emulsifier is PVA.

As stated hereinabove, the nanoparticle of the instant invention may comprise a hair regrowth agent covered or coated by the polymer. Hair regrowth agents which can be formulated in a nanoparticle of the present invention include, without limitation, proteins, peptides, small molecules, nucleic acids, or combinations thereof. In a particular embodiment, the hair regrowth agent is a small molecule. Hair regrowth agents include any agent that promotes hair regrowth and/or hair thickness. In some embodiments, the hair regrowth agent promotes the transition of vellus hair to terminal hair; increases vellus and/or terminal hair regrowth; maintains terminal hair regrowth; and/or prevents and/or inhibits miniaturization of terminal hairs. Examples of hair regrowth are provided, for example, in Gensure, R. (Chapter 4, “Pharmacological Treatment of Alopecia” in Alopecia, Ed. M. Ahmad, IntechOpen, 2018, DOI: 10.5772/intechopen.79656), incorporated by reference herein. These examples include, without limitation, spironolactone, minoxidil, finasteride, oral contraceptives, cyclosporine-A, glucocorticoids, Janus kinase (JAK) inhibitors (e.g., tofacitinib or ruxolitinib), bimatoprost, diphenylcyclopropenone (DPCP), androgen receptor antagonist, vitamin D analogs, parathyroid hormone antagonists, TGF-beta receptor antagonists, anti-fibrogenic factor, neurotrophic activator, histone deacetylase inhibitor (e.g., suberohydroxamic acid phenyl ester), and interleukin antibodies (e.g., tralokinumab or secukinumab). In a particular embodiment, the hair regrowth agent is selected from the group consisting of minoxidil, 5-alpha-reductase inhibitors (e.g., finasteride, dutasteride, alfatradiol, turosteride, bexlosteride, izonsteride, and epristeride), prostamides, cyclosporine-A, and prostaglandin F2α (PGF2α) analogs (e.g., bimatoprost, travoprost, latanoprost, dinoprost, carboprost, and tafluprost). In a particular embodiment, the hair regrowth agent is selected from the group consisting of minoxidil, finasteride, cyclosporine-A, and bimatoprost.

As stated hereinabove, the nanoparticle of the instant invention may comprise a repigmentation agent and/or an antioxidant covered or coated by the polymer. Antioxidants which can be formulated in a nanoparticle of the present invention include, without limitation, antioxidant enzymes, small molecule antioxidants, natural and synthetic, or combinations thereof. Antioxidants are substances which neutralize the activity of reactive oxygen species or inhibit the cellular damage done by the reactive species or their reactive byproducts or metabolites. The term “antioxidant” may also refer to compounds that inhibit, prevent, reduce or ameliorate oxidative reactions or compounds that inhibit reactions promoted by reactive oxygen species such as oxygen itself, oxygen free radicals, or peroxides. Examples of antioxidant enzymes include, but are not limited to: superoxide dismutase (e.g., SOD1), catalase, peroxidase, glutathione peroxidase, glutathione reductase, glutathione-S-transferase, methionine sulfoxide reductase, and hemeoxygenase. For example, the antioxidant enzyme superoxide dismutase (SOD), particularly, SOD1 (also called Cu/Zn SOD), is known to catalyze the dismutation of superoxide (O₂^{.-}). Examples of other antioxidants include, without limitation: Bcl-2 (B-cell lymphoma 2), plant derived antioxidants, vitamin E, vitamin C, ascorbyl palmitate, vitamin A, methionine, carotenoids, beta carotene, retinoids, xanthophylls, lutein, zeaxanthin, flavones, isoflavones, flavanones, flavonols, catechins, ginkgolides, anthocyanidins, proanthocyanidins, carnosol, carnosic acid, organosulfur compounds, allylcysteine, alliin, allicin, lipoic acid, omega-3 fatty acids, eicosapentaeneoic acid (EPA), docosahexaeneoic acid (DHA), tryptophan, arginine, isothiocyanates, quinones, ubiquinols, butylated hydroxytoluene (BHT), butylated hydroxyanisole (BHA), super-oxide dismutase mimetic (SODm), and coenzymes-Q. In a particular embodiment, the antioxidant is an antioxidant vitamin (e.g., Vitamin A, C, and/or E). In a particular embodiment, the antioxidant is an antioxidant enzyme, particularly catalase and/or methionine sulfoxide reductase (e.g., of mammalian, particularly human, origin). The antioxidant may be isolated from natural sources or prepared recombinantly.

The nanoparticles of the instant invention may be synthesized by known methods. Methods for synthesizing nanoparticles are provided in U.S. Pat. Nos. 7,332,159; 10,517,934; Adjei et al. (2014) Nanomedicine, 9:267-278; Singhal et al. (2013) Cell Death Dis., 4:e903; and Reddy et al. (2009) FASEB J., 23(5):1384-1395 (each of these references is incorporated by reference herein). In a particular embodiment, the nanoparticles are synthesized by a spray drying method. In a particular embodiment, the nanoparticles of the instant invention are synthesized by an emulsion solvent evaporation method. Any suitable emulsion may be used such as oil-in-water (o/w) emulsion, oil-in-oil (o/o) emulsion, water-in-oil (w/o) emulsion, water-in-oil-in-water (w/o/w) emulsion, or oil-in-water-in-oil (o/w/o) emulsion. In a particular embodiment, the nanoparticles of the instant invention are synthesized by a solid-in-oil-in-water emulsion method (e.g., Toorisaka, et al. (2018) J. Encapsul. Adsorp. Sci., 8:58-66; incorporated herein by reference). For example, a water and drug (e.g., hydrophilic drug (e.g., minoxidil)) in oil emulsion may be prepared and then lyophilized. The resultant solid may then be used in nanoparticle preparation. The nanoparticles may also be purified after synthesis by methods known in the art. For example, the nanoparticles may be purified by size exclusion chromatography (e.g., using a Sephacryl™ column), tangential flow filtration, and/or centrifugal filtration (e.g., using a molecular weight cutoff filter). In a particular embodiment, the nanoparticles are purified such that at least 95%, 96%, 97%, 98%, 99%, or more of undesired components are removed from the sample.

In accordance with another aspect of the instant invention, compositions comprising the nanoparticles of the instant invention are provided. In a particular embodiment, the composition is a topical composition (for application to the skin). The compositions of the instant invention comprise at least one nanoparticle and at least one carrier (e.g., a carrier acceptable for topical delivery (e.g., a carrier acceptable for skin application; e.g., a pharmaceutically and/or cosmetically acceptable carrier). The topical compositions of the present invention may be made into a wide variety of product types such as, without limitation, liquids, lotions, powders, creams, salves, gels, foams, milky lotions, sticks, sprays (e.g., pump spray), aerosols, ointments, pastes, mousses, dermal patches, adhesives (e.g., adhesive tape), bandages, pad, scaffold, nanofibers, films, cleansing agent, controlled release devices, and other equivalent forms. In a particular embodiment, the composition is a lotion or cream product. In some embodiments, the composition is a hair care or body care product such as, without limitation, a hair shampoo, hair conditioner, hair foam, hair spray, lotion, gel, cream, ointment, soap, powder, or a sprayable powder.

Acceptable carriers can be, without limitation, sterile liquids, such as water (may be deionized), alcohol (e.g., ethanol, isopropanol, benzyl alcohol), oils (including those of petroleum, animal, vegetable or synthetic origin, such as peanut oil, soybean oil, mineral oil, sesame oil and the like), and other organic compounds or copolymers. Water or aqueous saline solutions and aqueous dextrose and glycerol solutions may also be employed as carriers. Suitable carriers and other agents of the compositions of the instant invention are described in “Remington's Pharmaceutical Sciences” by E. W. Martin (Mack Pub. Co., Easton, PA) and “Remington: The Science and Practice of Pharmacy” by Alfonso R. Gennaro (Lippincott Williams & Wilkins) (each of the foregoing references being incorporated herein by reference). Additional general types of acceptable topical carriers include, without limitation, emulsions (e.g., microemulsions and nanoemulsions), gels (e.g., an aqueous, alcohol, alcohol/water, or oil (e.g., mineral oil) gel using at least one suitable gelling agent (e.g., natural gums, acrylic acid and acrylate polymers and copolymers, cellulose derivatives (e.g., hydroxymethyl cellulose and hydroxypropyl cellulose), and hydrogenated butylene/ethylene/styrene and hydrogenated ethylene/propylene/styrene copolymers), solids (e.g., a wax-based stick, soap bar composition), or powder (e.g., bases such as talc, lactose, starch, and the like), spray, and liposomes (e.g., unilamellar, multilamellar, and paucilamellar liposomes, optionally containing phospholipids). The acceptable carriers also include stabilizers, penetration enhancers, chelating agents (e.g., EDTA, EDTA derivatives (e.g., disodium EDTA and dipotassium EDTA), iniferine, lactoferrin, and citric acid), and excipients. Protocols and procedures which facilitate formulation of the topical compositions of the invention can be found, for example, in Cosmetic Bench Reference (Cosmetics & Toiletries, Allured Publishing Corporation, Illinois) and in International Cosmetic Ingredient Dictionary and Handbook (15^{th} Ed.) (each of the foregoing references being incorporated herein by reference).

The topical composition of the instant invention may be aqueous or anhydrous. In a particular embodiment, the composition is anhydrous (e.g., anhydrous serum). In a particular embodiment, the composition is silicone-based (e.g., comprising polysilicone-11 and/or cyclopentasiloxane (e.g., Gransil GCM-5)). The topical composition of the instant invention may comprise the nanoparticles in a wide range of concentration. In a particular embodiment, the topical composition comprises from about 0.001% to about 20.0% nanoparticles, about 0.001% to about 10.0% nanoparticles, about 1.0% to about 20.0% nanoparticles, about 1.0% to about 5.0% nanoparticles, about 0.001% to about 5.0% nanoparticles, about 0.001% to about 1.0% nanoparticles, or about 0.005% to 0.5% nanoparticles (e.g., by weight). In a particular embodiment, the topical composition comprises about 5% nanoparticles (e.g., by weight). In a particular embodiment, the topical composition comprises at least 0.5% nanoparticles, at least 1% nanoparticles, at least about 2.5% nanoparticles, or at least about 5% nanoparticles (e.g., by weight).

As stated hereinabove, the compositions of the instant invention may further comprise at least one other agent (e.g., therapeutic agent) in addition to the nanoparticles. Alternatively, the other agent (e.g., therapeutic agent) may be contained within another separate composition from the nanoparticles of the instant invention. The compositions may be administered at the same time or at different times (e.g., sequentially). In a particular embodiment, to achieve sequential delivery, the product can be developed in the form of layers (e.g., in bandage or scaffold). Additional agents (e.g., therapeutic agents) that may be included in the compositions of the instant invention include, without limitation: additional hair regrowth agents (e.g., other agents not contained within the nanoparticles) and/or antioxidants. The agents may be incorporated in oil phase or water phase or in both (e.g., of a topical cream or lotion).

These nanoparticles may be employed therapeutically under the guidance of a physician or other healthcare professional or self-administered by the subject/patient. The pharmaceutical preparation comprising the nanoparticles of the invention may be conveniently formulated for administration with an acceptable medium such as water, buffered saline, ethanol, polyol (for example, glycerol, propylene glycol, liquid polyethylene glycol and the like), dimethyl sulfoxide (DMSO), oils, detergents, suspending agents or suitable mixtures thereof. The concentration of nanoparticles in the chosen medium may depend on the hydrophobic or hydrophilic nature of the medium, as well as the size, enzyme activity, and other properties of the nanoparticles. Solubility limits may be easily determined by one skilled in the art.

As used herein, “acceptable medium” or “carrier” includes any and all solvents, dispersion media and the like which may be appropriate for the desired route of administration of the preparation, as exemplified in the preceding discussion. In a particular embodiment, the carrier is for topical application and is a pharmaceutically acceptable carrier or a cosmetically acceptable carrier. The use of such media for active substances is known in the art. Except insofar as any conventional media or agent is incompatible with the nanoparticles to be administered, its use in the pharmaceutical preparation is contemplated.

The dose and dosage regimen of a nanoparticle according to the invention that is suitable for administration to a particular subject may be varied considering the patient's age, sex, weight, general medical condition, and the specific condition for which the nanoparticle is being administered and the severity thereof. The route of administration of the nanoparticle, the pharmaceutical carrier with which the nanoparticle is combined, and the nanoparticle's biological activity may also be considered.

Selection of a suitable pharmaceutical preparation may also depend upon the mode of administration chosen. For example, the nanoparticles of the invention may be administered topically. In these instances, the pharmaceutical preparation comprises the nanoparticles dispersed in a medium that is compatible with the site of administration (e.g., skin). In a particular embodiment, the nanoparticles may also be injected into skin layers either using needle or diffused through the skin layers using ultrasound/UV rays/permeability enhancers or physical and mechanical techniques. As explained hereinabove, pharmaceutical preparations for topical administration are known in the art. The lipophilicity of the nanoparticles or the pharmaceutical preparation in which they are delivered may be increased so that the molecules can arrive at their target location. Methods for increasing the lipophilicity of a molecule are known in the art.

Pharmaceutical compositions containing a nanoparticle of the present invention as the active ingredient in intimate admixture with a pharmaceutical carrier can be prepared according to conventional pharmaceutical compounding techniques. The carrier may take a wide variety of forms depending on the form of preparation desired for administration, e.g., topically. A pharmaceutical preparation of the invention may be formulated in dosage unit form for ease of administration and uniformity of dosage. Dosage unit form, as used herein, refers to a physically discrete unit of the composition appropriate for the subject using the nanoparticles of the instant invention. Each dosage should contain a quantity of active ingredient calculated to produce the desired effect in association with the selected carrier. Procedures for determining the appropriate dosage unit are well known to those skilled in the art. Appropriate concentrations for alleviation of a particular pathological condition may be determined by dosage concentration curve calculations, as known in the art.

In accordance with the present invention, the appropriate dosage unit for the administration of nanoparticles may be determined by evaluating the toxicity of the molecules in animal models. Various concentrations of nanoparticle pharmaceutical preparations may be administered to mice or other mammals, and the minimal and maximal dosages may be determined based on the beneficial results and side effects observed as a result of the treatment. Appropriate dosage unit may also be determined by assessing the efficacy of the nanoparticles treatment in combination with other standard drugs. The dosage units of nanoparticles may be determined individually or in combination with each treatment according to the effect detected.

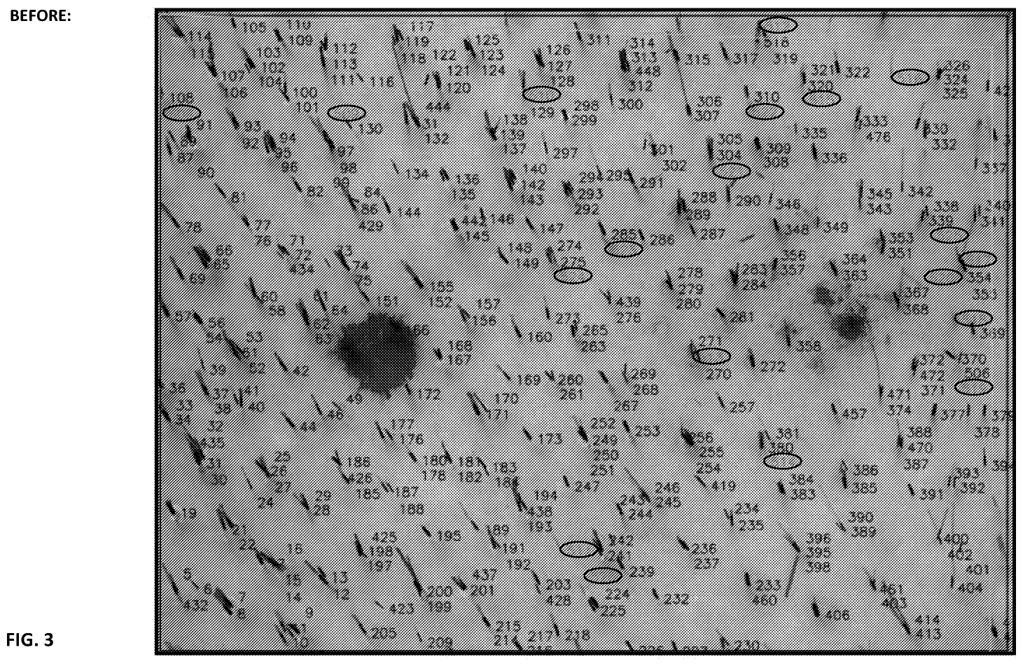

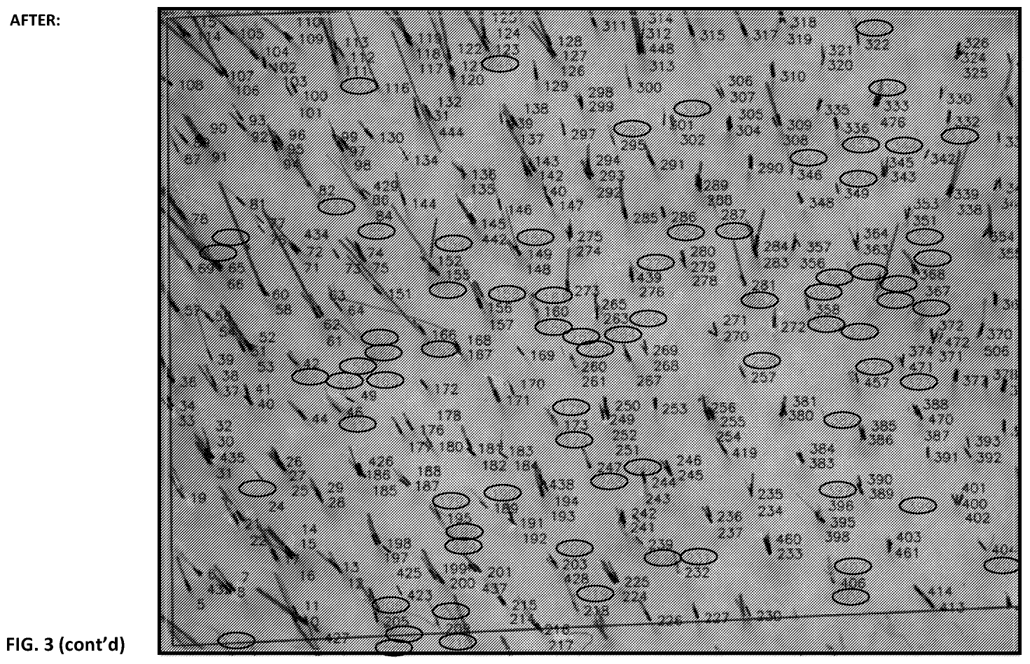

The compositions comprising the nanoparticles may be administered at appropriate intervals. In certain embodiments, the composition will be administered frequently until the pathological symptoms are reduced or alleviated, after which the dosage and/or frequency may be reduced to a maintenance level. The appropriate interval in a particular case would normally depend on the condition of the patient. The compositions may also be administered “as needed”. In certain embodiments, the composition is administered more than once daily, twice daily, daily, or once every 1 day, 2 days, 3 days, 4 days, 5 days, 6 days, 7 days or more. Herein, in a trial of 3 men suffering from alopecia, finasteride loaded nanoparticles were applied once daily to the scalp (see Example). In all three subjects, hair regrowth was observed after just one month in excess of what would be expected from topically administered Minoxidil after one year or comparable to orally administered finasteride after two years (see, e.g., FIG. 3, Table 1), thereby demonstrating unexpectedly superior results with the nanoparticles of the instant invention.

### Definitions

The following definitions are provided to facilitate an understanding of the present invention:

The singular forms “a,” “an,” and “the” include plural referents unless the context clearly dictates otherwise.

As used herein, the term “polymer” denotes molecules formed from the chemical union of two or more repeating units or monomers. The term “block copolymer” most simply refers to conjugates of at least two different polymer segments, wherein each polymer segment comprises two or more adjacent units of the same kind.

The term “treat” as used herein refers to any type of treatment that imparts a benefit to a patient afflicted with a disease, including improvement in the condition of the patient (e.g., in one or more symptoms), delay in the progression of the condition, etc.

As used herein, the term “prevent” refers to the prophylactic treatment of a subject who is at risk of developing a condition resulting in a decrease in the probability that the subject will develop the condition.

As used herein, the term “subject” refers to an animal, particularly a mammal, particularly a human.

A “therapeutically effective amount” of a compound or a pharmaceutical composition refers to an amount effective to prevent, inhibit, treat, or lessen the symptoms of a particular disorder or disease. The treatment of inflammation or infection herein may refer to curing, relieving, and/or preventing the inflammation or infection, the symptom(s) of it, or the predisposition towards it.

As used herein, the term “therapeutic agent” refers to a chemical compound or biological molecule including, without limitation, nucleic acids, peptides, proteins, and antibodies that can be used to treat a condition, disease, or disorder or reduce the symptoms of the condition, disease, or disorder.

As used herein, the term “small molecule” refers to a substance or compound that has a relatively low molecular weight (e.g., less than 4,000, less than 2,000, particularly less than 1 kDa or 800 Da). Typically, small molecules are organic, but are not proteins, polypeptides, or nucleic acids, though they may be amino acids or dipeptides.

As used herein, the term “amphiphilic” means the ability to dissolve in both water and lipids/apolar environments. Typically, an amphiphilic compound comprises a hydrophilic portion and a hydrophobic portion. “Hydrophobic” designates a preference for apolar environments (e.g., a hydrophobic substance or moiety is more readily dissolved in or wetted by non-polar solvents, such as hydrocarbons, than by water). As used herein, the term “hydrophilic” means the ability to dissolve in water.

“Pharmaceutically acceptable” indicates approval by a regulatory agency of the Federal or a state government or listed in the U.S. Pharmacopeia or other generally recognized pharmacopeia for use in animals, and more particularly in humans.

A “carrier” refers to, for example, a diluent, adjuvant, preservative (e.g., Thimersol, benzyl alcohol), anti-oxidant (e.g., ascorbic acid, sodium metabisulfite), solubilizer (e.g., Polysorbate 80), emulsifier, buffer (e.g., Tris HCl, acetate, phosphate), bulking substance (e.g., lactose, mannitol), excipient, auxiliary agent or vehicle with which an active agent of the present invention is administered. Pharmaceutically or cosmetically acceptable carriers can be sterile liquids, such as water and oils, including those of petroleum, animal, vegetable or synthetic origin, such as peanut oil, soybean oil, mineral oil, sesame oil and the like. Water or aqueous saline solutions and aqueous dextrose and glycerol solutions are preferably employed as carriers, particularly for injectable solutions. The compositions can be incorporated into particulate preparations of polymeric compounds such as polylactic acid, polyglycolic acid, etc., or into liposomes or micelles. Such compositions may influence the physical state, stability, rate of in vivo release, and rate of in vivo clearance of components of a pharmaceutical composition of the present invention. The pharmaceutical composition of the present invention can be prepared, for example, in liquid form, or can be in dried powder form (e.g., lyophilized). Suitable pharmaceutical carriers are described in “Remington's Pharmaceutical Sciences” by E. W. Martin (Mack Publishing Co., Easton, PA); Gennaro, A. R., Remington: The Science and Practice of Pharmacy, (Lippincott, Williams and Wilkins); Liberman, et al., Eds., Pharmaceutical Dosage Forms, Marcel Decker, New York, N.Y.; and Kibbe, et al., Eds., Handbook of Pharmaceutical Excipients, American Pharmaceutical Association, Washington.

As used herein, the term “purified” or “to purify” refers to the removal of contaminants or undesired compounds from a sample or composition. For example, purification can result in the removal of from about 70 to 90%, up to 100%, of the contaminants or undesired compounds from a sample or composition. In certain embodiments, at least 90%, 93%, 95%, 96%, 97%, 98%, 99%, 99.5%, or more of undesired compounds from a sample or composition are removed from a preparation.

The following example provides illustrative methods of practicing the instant invention and is not intended to limit the scope of the invention in any way.

### Example

### Nanoparticle Synthesis

Nanoparticles were prepared using an emulsion solvent evaporation method. For finasteride loaded particles, the polymer solution was formed by dissolving PLGA (27.6 mg/mL), dimethyl tartrate (2.76 mg/mL) and finasteride (3 mg/mL) into ethyl acetate. An aqueous solution was formed by dissolving PVA (30 mg/mL) into deionized water and then saturating the solution with ethyl acetate. An emulsion was then formed by homogenizing the polymer solution into the aqueous solution at a volume ratio of 1:2, until the desired particle size was achieved (˜450 nm). The ethyl acetate was then removed by rotary evaporator. The resulting particles were then purified of residual PVA via tangential flow filtration. The purified particles were then lyophilized and analyzed for size, polydispersity index, and drug loading and stored at −20° C. until use. For these particles, typical values were size ˜400-500 nm, PDI <0.20, and drug loading ˜5-20 μg/mg.

For cyclosporine-A loaded particles, the polymer solution was formed by dissolving PLGA (27.6 mg/mL), dimethyl tartrate (2.76 mg/mL) and cyclosporine-A (6.9 mg/mL) into ethyl acetate. An aqueous solution was formed by dissolving PVA (30 mg/mL) into deionized water and then saturating the solution with ethyl acetate. An emulsion was then formed by homogenizing the polymer solution into the aqueous solution at a volume ratio of 1:2, until the desired particle size was achieved (˜250 nm). The ethyl acetate was then removed by rotary evaporator and the resulting particles were then purified of residual PVA via tangential flow filtration. The purified particles were then lyophilized and analyzed for size, polydispersity index, and drug loading and stored at −20° C. until use. For these particles, typical values were size ˜220-300 nm, PDI <0.20, and drug loading ˜100-300 μg/mg.

### Lotion Preparation

To prepare lotion containing the nanoparticles, the lyophilized particles were weighed out and mixed into Gransil 314 (a silicone blend of about 15% super high viscosity silicone (dimethicone) with cyclopentasiloxane) at a ratio of 1:10 w/w. This mixture is gently stirred and allowed to sit until dry lyophilized particles are no longer present. Then the mixture is geometrically diluted into Gransil GCM-5 until the desired particle concentration is achieved (e.g., 5% for finasteride particles used in the human trial in FIG. 3).

### Extended Release Experiment

FIGS. 1 and 2 were generated using finasteride and cyclosporine-A loaded particles, respectively. Lyophilized particles were resuspended in deionized water and allowed to incubate for a period of time. To collect a sample, particles were centrifuged and the supernatant was taken for analysis. The particles were then resuspended in fresh deionized water and continued to incubate. The supernatant was then analyzed for finasteride or cyclosporine-A concentration using UPLC and compared to a known standard sample of finasteride.

### Efficacy Study

A pilot study was performed to evaluate the efficacy of finasteride loaded particles in a topical formulation for the treatment of male pattern baldness (androgenetic alopecia) over a period of one month. The lotion comprising finasteride loaded particles (5%, yielding approximately finasteride 0.25% by weight) was applied once a day at night on the vertex (crown) area of the scalp. The subjects (n=3) were males between the ages of 30-65 in good health with mild to moderate male pattern hair loss (androgenic alopecia) with ongoing hair loss for at least 1 year and a hair loss pattern of Norwood Scale Types 3V, 4, 5, 5V with a density of visible thinning of hair in the vertex area with scalp visible.

Automated hair assessment was performed in a standardized 2 cm² area (marked with virtual tattoo microdots) using the Leviacam® camera and software (TrichoLAB) to analyze the following: hair counts, individual hair tracking, number of anagen hairs, number of telogen hairs, telogen ratio (growth rate <0.1 mm/d), density, diameter, and hair distribution. Digital photography of the vertex area of the scalp before and after allows for hair to hair matching to determine the effect on hair growth.

### Results

The in vitro release of drug from finasteride and cyclosporine-A loaded particles into deionized water was studied. As seen in FIG. 1, finasteride loaded particles showed measurable release of finasteride through at least day 4, the end point of the study. As seen in FIG. 2, cyclosporine-A loaded particles showed measurable release of drug through at least day 17, the end point of the study.

To demonstrate the efficacy of the finasteride loaded particles, a lotion comprising the finasteride loaded particles was administered daily to the scalp of subjects with androgenetic alopecia. FIG. 3 provides exemplary images of hair regrowth in one subject after one month of finasteride loaded nanoparticles. The photo on the left of FIG. 3 shows the baseline amount of hair, with circled numbers indicating individual hairs that were no longer present at month 1. The photo on the right of FIG. 3 shows the scalp at 1 month with circled numbers indicating hairs that appeared after start of application. The average increase in hair density was calculated. In all three subjects, hair regrowth was observed after just one month in excess of what would be expected from topically administered Minoxidil after one year or comparable to orally administered finasteride after two years (see, Table 1). These results demonstrate unexpectedly superior results (e.g., with regard to increasing hair density) with the nanoparticles of the instant invention.

TABLE 1
Comparison of finasteride loaded nanoparticles to commercially
available alopecia treatments. Finasteride loaded nanoparticles
data comes from human subjects suffering from alopecia (N = 3).

| | | | Average Increase in |
|---|---|---|---|
| Treatment | Time Frame | | Hair Density |
| Rogaine (5% Topical Minoxidil) | 12 | Months | 5% |
| Propecia (Finasteride Oral Tablet) | 24 | Months | 16% |
| Finasteride Loaded Nanoparticles | 1 | Month | 15% |

A number of publications and patent documents are cited throughout the foregoing specification in order to describe the state of the art to which this invention pertains. The entire disclosure of each of these citations is incorporated by reference herein.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method of treating, inhibiting, and/or preventing hair loss and/or regrowing and/or thickening hair in a subject in need thereof, said method comprising topically administering at least one nanoparticle to the skin of the subject,
wherein said nanoparticle comprises at least one biodegradable polymer and at least one hair regrowth agent.

2. The method of claim 1, wherein said hair regrowth agent is minoxidil, finasteride, cyclosporine-A, a Janus kinase (JAK) inhibitor, or bimatoprost.

3. The method of claim 1, wherein said hair loss is caused by alopecia, alopecia areata, androgenetic alopecia, hypotrichosis, or hair miniaturization.

4. The method of claim 1, further comprising administering at least one other hair regrowth agent or at least one antioxidant.

5. The method of claim 1, wherein said biodegradable polymer is poly(lactide-co-glycolide).

6. The method of claim 1, wherein said nanoparticle further comprises at least one plasticizer.

7. The method of claim 6, wherein said plasticizer is dimethyl tartrate.

8. The method of claim 1, wherein said nanoparticle further comprises at least one surfactant or emulsifier.

9. The method of claim 8, wherein said surfactant or emulsifier is polyvinyl alcohol.

10. The method of claim 1, wherein said nanoparticles are formulated by an emulsion solvent evaporation method or a solid-in-oil-in-water emulsion.

11. The method of claim 1, wherein said biodegradable polymer is poly(DL-lactide-co-glycolide).

12. The method of claim 11, wherein said nanoparticles further comprise at least one plasticizer.

13. The method of claim 12, wherein said plasticizer is dimethyl tartrate.

14. The method of claim 13, wherein said nanoparticle further comprises at least one surfactant or emulsifier.

15. The method of claim 14, wherein said surfactant or emulsifier is polyvinyl alcohol.

16. The method of claim 15, wherein said hair regrowth agent is finasteride.

17. The method of claim 1, further comprising administering at least one repigmentation agent.

18. The method of claim 17, wherein said repigmentation agent is an antioxidant.

19. The method of claim 18, wherein said antioxidant is catalase.

20. A method of treating, inhibiting, and/or preventing graying hair and/or reducing the amount of gray hair in a subject in need thereof, said method comprising topically administering at least one nanoparticle to the skin of the subject,
wherein said nanoparticle comprises at least one biodegradable polymer and at least one repigmentation agent.
